(19) United States
(12) Patent Application Publication
(10) Pub. No.: US 2017/0333407 A1
(43) Pub. Date: Nov. 23, 2017

(54) METHOD FOR TREATING UNDERACTIVE BLADDER SYNDROME

(71) Applicant: William Beaumont Hospital, Royal Oak, MI (US)

(72) Inventors: Michael Chancellor, Pittsburg, PA (US); David D. Chancellor, Pittsburg, PA (US)

(21) Appl. No.: 15/524,000

(22) PCT Filed: Oct. 30, 2015
(86) PCT No.: PCT/US15/58238

§ 371 Date: May 3, 2017

Related U.S. Application Data
(60) Provisional application No. 62/074,187, filed on Nov. 3, 2014.

(51) Int. Cl. A61K 31/439

(52) U.S. Cl. CPC A61K 31/439

(57) ABSTRACT

Disclosed are nonlimiting embodiments comprising novel methods for treating underactive bladder in a subject, including administering an effective amount of a pharmaceutical composition comprising an M1-selective muscarinic agonist to the subject. In some embodiments, the M1-selective muscarinic agonist is cevimeline.

### CROSS REFERENCE TO RELATED APPLICATION

This PCT application claims the benefit of U.S. Provisional application No. 62/074,187, filed on Nov. 3, 2014. The entire contents of the aforementioned application are hereby incorporated by reference in their entirety.

### TECHNICAL FIELD

Some embodiments relate generally to improving bladder function. More specifically, some embodiments relate to improving bladder emptying in subjects having an underactive bladder disorder or syndrome.

### BACKGROUND

Underactive bladder disorder or syndrome (UAB) involves deterioration of bladder function and include unpleasant symptoms such as incomplete bladder emptying. Other common symptoms of UAB include increased urination frequency, urgency, hesitancy, a weak urine stream, reduced urine flow, urine stream splitting, an intermittent urine stream, difficulty starting and/or stopping urination, incontinence, nocturia, straining to urinate, terminal dribbling of urine, a feeling of incomplete bladder emptying after urination, bladder pain, urethral pain, and recurrent urinary infections. Causes of UAB can vary and include, for example, a weak bladder detrusor muscle, damage to sensory and/or motor nerves that innervate the bladder, central nervous system disorders, and bladder outlet obstruction.

For the treatment of UAB, drugs that enhance the contractility of the bladder detrusor or reduce urethral resistance through the relaxation of the urethral sphincter are used. For example, cholinergic agents, such as bethanechol and acetylcholinesterase inhibitors, such as distigmine, are used as drugs for enhancing the contractility of the bladder detrusor. However, bethanechol also contributes to the contraction of the bladder detrusor at the urine collection period, which causes damage to the urine collection function of the bladder, and at the same time, has side effects such as lacrimation, perspiration, gastrointestinal disorders, and abdominal pain. Therefore, it is contraindicated for pregnant women, and patients suffering with peptic ulcer, organic intestinal tract obstruction, asthma, and hyperthyroidism.

Acetylcholinesterase inhibitors, for example, distigmine and neostigmine, have also been used. Since acetylcholinesterase inhibitors enhance the activity of acetylcholine released from the pelvic nerve endings in urination to enhance the contraction of the bladder detrusor in urination, they are considered excellent drugs when the physiological mechanism of micturition is taken into consideration. However, since distigmine contracts the bladder detrusor and causes the contraction of the urethral sphincter due to a potent nicotine-like activity thereof to increase urethral resistance, voiding efficiency is not good and effects in terms of clinical application is insufficient. Additionally, the risk of high-pressure voiding has also been identified.

Drugs for relaxing the urethral sphincter and reducing urethral resistance, α1 receptor antagonists, such as tamsulosin, prazosin, alfuzosin, naftopidil, and urapidil, also have been used. These antagonists are reported to be effective for the amelioration of subjective symptoms, such as feeling of residual urine and nocturia. However, since there are antihypertensive effects including orthostatic hypotension etc. as a side effect care should be taken for administration thereof. Additionally, there has been no report demonstrating satisfactory effects on UAB.

The long-term effects of UAB can lead to other conditions, as well. For example, urine left behind in the bladder may lead to urinary tract infections that can be extraordinarily painful and, if they become chronic, can lead to kidney damage. Sediments can also accumulate in the bladder, forming bladder stones and blood in the urine. In severe cases, urine left behind in the bladder can build up to a level that causes reflux up the ureters and may cause kidney damage.

Currently, no medications or therapies have proven effective in the long-term treatment of UAB and no known cure exists. Consequently, patients who suffer from UAB are usually managed with intermittent self-catheterization, indwelling catheters, wearing absorbent undergarments, and/or riskier surgical procedures such as suprapubic catheter or urinary diversion with urostomy.

Therefore, it would be advantageous to reduce these bladder issues using a less-invasive treatment method, such as, for example, the use of appropriate medication. These needs and other needs are satisfied by the methods of the present invention.

### SUMMARY

In one aspect, a method for treating an underactive bladder syndrome in a subject in need thereof is provided. The method comprises administering an effective amount of a pharmaceutical composition comprising an M1-selective muscarinic agonist to the subject.

In another aspect, a method for decreasing a residual urine volume in a subject after urination is provided. The method comprises administering an effective amount of a pharmaceutical composition comprising an M1-selective muscarinic agonist to the subject.

In still another aspect, a method for increasing bladder emptying in a subject is provided. The method comprises administering an effective amount of a pharmaceutical composition comprising an M1-selective muscarinic agonist to the subject.

In a further aspect, a method for increasing a urine flow rate in a subject is provided. The method comprises administering an effective amount of a pharmaceutical composition comprising an M1-selective muscarinic agonist to the subject.

Additional aspects will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

In the following description, numerous specific details are given to provide a thorough understanding of the embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment,” “an embodiment,” or “embodiments” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

Unless indicated otherwise, when a range of any type is disclosed or claimed, it is intended to disclose or claim individually each possible number that such a range could reasonably encompass, including any sub-ranges encompassed therein. Moreover, when a range of values is disclosed or claimed, which Applicants intend to reflect individually each possible number that such a range could reasonably encompass, Applicants also intend for the disclosure of a range to reflect, and be interchangeable with, disclosing any and all sub-ranges and combinations of sub-ranges encompassed therein. Accordingly, Applicants reserve the right to provision out or exclude any individual numbers or ranges, including any sub-ranges or combinations of sub-ranges within the group, if for any reason the Applicants choose to claim less than the full measure of the disclosure, for example, to account for a reference that Applicants are unaware of at the time of the filing of the application.

The Abstract of this disclosure is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure”. Therefore, the Abstract of this disclosure is not intended to be used to construe the scope of the claims or to limit the scope of subject matter that is disclosed herein. Moreover, any headings that may be employed herein are also not intended to be used to construe the scope of the claims or to limit the scope of the subject matter that is disclosed herein.

### I. Definitions

The present invention can improve the symptoms that are associated with UAB. The term “improvement,” “improve,” or “improved” includes improvement or remission from one or more symptoms of UAB. Improvement from the symptoms associated with UAB is manifested by increased bladder emptying, decreased residual bladder volume after urination, increased urine flow rate, decreased urination urgency, decreased bladder pain, decreased uretheral pain, decreased urinary incontinence, and the like. Improvement not only includes remission from these symptoms, but may also include alleviation of symptoms, remission of deterioration of symptoms, prevention of manifestation, improvement of the Quality Of Life (QOL), and the like.

As used herein, the term “active ingredient” means a compound that, when formulated into a pharmaceutical composition, is effective for treating underactive bladder syndrome.

As used herein, the terms “administer,” “administered,” or “administering,” to a subject include dispensing, delivering, or applying a pharmaceutical composition to a subject by any suitable route for delivery.

The term “co-administration,” “administered in combination with,” and their grammatical equivalents, as used herein, encompass administration of two or more agents to a subject so that both agents and/or their metabolites are present in the subject at the same time. Co-administration includes simultaneous administration in separate compositions, administration at different times in separate compositions, or administration in a composition in which both agents are present.

The term “agonist” as used herein refers to a compound having the ability to initiate and/or enhance (i.e., to “agonize”) a biological function of a target protein, whether by increasing the activity or expression of the target protein. Accordingly, the term “agonist” is defined in the context of the biological role of the target polypeptide. While preferred agonists herein specifically interact with (e.g. bind to) the target, compounds that initiate or enhance a biological activity of the target polypeptide by interacting with other members of the signal transduction pathway of which the target polypeptide is a member are also specifically included within this definition.

As used herein, the term “bladder” is synonymous with “urinary bladder.”

As used herein, the term “compounds” or “the compound” mean M1-selective muscarinic receptor agonists (for example, cevimeline).

As used herein, the term “detrusor underactivity” means a contraction of reduced strength and/or duration, resulting in prolonged bladder emptying, and/or failure to achieve complete bladder emptying within a normal time span.

As used herein, the term “micturition” is synonymous with the terms “voiding” and “urination.”

Among the muscarinic receptors M1-M5, the terms “M1-selective muscarinic receptor agonist” and “M1-selective muscarinic agonist” are used herein to refer to a compound or a pharmaceutical composition that selectively agonizes at least the M1 muscarinic receptor. An M1-selective muscarinic agonist does not necessarily interact with only the M1 muscarinic receptor but may also agonize, or selectively agonize, other muscarinic receptors (i.e., M2-M5) either directly or indirectly, including through positive or negative feedback loops. M1-selective muscarinic agonists or M1 allosteric modulators may increase the M1 feedback loop on the nerves innervating the urinary bladder and thereby extend, increase, and/or maintain the duration of bladder contraction and/or improve bladder emptying.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier” or “pharmaceutically acceptable excipient” includes all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the pharmaceuticals compositions is contemplated. Supplementary active ingredients can also be incorporated into the compositions.

As used herein, the term “pharmaceutical composition” refers to a mixture of the compound with other chemical components, such as diluents, lubricants, bulking agents, desentegrant, or carriers. The pharmaceutical composition facilitates administration of the compound to a subject. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, and the like.

“Prodrug” is meant to indicate a compound that may be converted under physiological conditions or by solvolysis to a biologically active compound described herein. Thus, the term “prodrug” refers to a precursor of a biologically active compound that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino, or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate, and benzoate derivatives of an alcohol or acetamide, formamide, and benzamide derivatives of an amine functional group in the active compound and the like.

As used herein, the terms “treat,” “treated,” “treating,” or “treatment” include the diminishment or alleviation of at least one symptom associated or caused by the syndrome, state, disorder, or disease being treated. In certain embodiments, the treatment includes the preventing of the induction of a disorder that would in turn diminish or alleviate at least one symptom associated or caused by the disorder being treated. For example, treatment can be diminishment of one or several symptoms of a disorder or complete eradication of a disorder. For prophylactic benefit, the compositions may be administered to a patient at risk of developing a particular disease, or to a patient reporting one or more of the physiological symptoms of a disease, even though a diagnosis of this disease may not have been made.

“Subject” refers to an animal, such as a mammal, for example a human. The methods described herein can be useful in both human and veterinary applications. In some embodiments, the subject is a mammal, and in some embodiments, the subject is human.

As used herein, the term “sustained-release” refers to the manner in which an active ingredient of a pharmaceutical composition is released from, for example, a tablet, such that the tablet is capable of releasing the active ingredient to the subject for a prolonged period. In certain embodiments, the sustained-release component is any suitable hydrophobic material that delays the release of a compound or a pharmaceutical composition into the subject. In certain embodiments, the hydrophobic material includes, but is not limited to, one or more of fatty alcohols, glyceryl monostearate, glyceryl behenate, and the like. In some embodiments, the sustained-release formulation is an orally disintegrating tablet or capsule. In some embodiments, the sustained-release component is configured to deliver a compound or a pharmaceutical composition to a subject and provides a steady-state concentration of the compound or the pharmaceutical composition to the subject over a period of about 1 hour to about 24 hours, about 1 hour to about 18 hours, about 1 hour to about 12 hours, or about 1 hour to about 6 hours.

As used herein, the term “rapid-release” refers to the release of an active ingredient of a pharmaceutical composition from, for instance, a tablet, in a short period. In certain embodiments, the rapid-release component may include a polymeric material. In particular embodiments, the rapid-release component is a polymer material comprising a film coating. In embodiments, the polymeric material of the film coating may comprise one or more of hydroxypropylmethylcellulose, hydroxypropylcellulose, ethylcellulose, methacrylic acid copolymers, and the like. In still other embodiments, pharmaceutical compositions may include any suitable rapid-release orally disintegrating tablet or capsule formulation, as would be known to those of skill in the art. In embodiments, the rapid-release component delivers a compound or a pharmaceutical composition to a subject and provides a maximal concentration Cₘₐₓ of the compound or the pharmaceutical composition to the subject in a time range of about 1 minute to about 100 minutes, about 5 minutes to about 60 minutes, about 10 minutes to about 30 minutes, or about 20 minutes.

The term “effective amount” refers to the amount of a compound or pharmaceutical composition described herein that is sufficient to effect the intended application including but not limited to treatment of a disorder, symptom, or syndrome; here, underactive bladder syndrome. The effective amount may vary depending upon the intended application, or the subject and condition being treated, e.g., the weight and age of the subject, the severity of the disorder or symptom, the manner of administration and the like, which can readily be determined by one of ordinary skill in the art. The term also applies to a dose that will induce a particular response in target cells, e.g. urothelial cells. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “underactive bladder syndrome” is synonymous with underactive bladder, lazy bladder, shy bladder, urinary retention, paralysis of the bladder, atonic bladder, incomplete bladder emptying, detrusor areflexia, detrusor underactivity, and chronic bothersome inability to empty the urinary bladder. Underactive bladder may be associated with one or more causes, including a neurogenic disorder, a brain disease, a surgical injury, a medication, a result of aging, an infection, a psychological condition, or a physical defect.

Examples of causative diseases of neurogenic underactive bladder include: peripheral nerve disorders such as diabetes, disc hernia, spinal canal stenosis, Guillain-Barre syndrome, and herpes zoster-induced peripheral neuritis; spinal cord diseases, for example, supranuclear spinal cord injury, spinal cord tumor, cervical spondylosis, vascular diseases of the spinal cord, spina bifida, myelomeningocele and tethered cord syndrome; and brain diseases, such as dementia, cerebrovascular diseases, Parkinson's disease, spinocerebellar degeneration, olivopontocerebellar atrophy (OPCA), Shy-Drager syndrome, brain tumor, multiple sclerosis, cerebral trauma and encephalitis etc. In some cases, underactive bladder is caused by surgical injury of pelvic nerve, hypogastric nerve, or pudendal nerve controlling voiding functions after surgical operations of pelvic viscera (uterine cancer or rectal cancer).

Examples of drug-induced underactive bladder include underactive bladder developed by anticholinergic drugs, i.e., drugs that inhibit release of acetylcholine and other factors.

Additionally, aged people generally exhibit dysuria caused by weakened bladder activity, and as a result, age-related underactive bladder becomes an important problem in an aging society. Other examples of factors that cause underactive bladder include physical defects such as lower urinary tract obstruction caused by prostatic hyperplasia, bladder neck contracture, or uterine prolapse, infections such as cystitis and urethritis, and psychological conditions such as stress.

As used herein, the terms “urinary retention” and “incomplete bladder emptying” mean the inability to empty the urinary bladder with voiding resulting in residual urine volume in the bladder after urination. In some embodiments, the M1-selective muscarinic agonists may act to decrease residual urine volume in the bladder after urination. In some embodiments, the M1-selective muscarinic agonists may increase bladder emptying. Increased bladder emptying decreases the amount of residual urine volume in the bladder after urination.

As used herein, the term “increased urine flow rate” means an increase in the volume urine voided during a given period of time. The increase is as compared to a subject that has not been administered or treated with an M1-selective muscarinic agonist.

As used herein, the term “decreased urination frequency” means an increase in the amount of time between urination or urination attempts for a subject needing to void. The increase is as compared to a subject that has not been administered or treated with an M1-selective muscarinic agonist.

As used herein, the term “decreased urination urgency” refers to a subject that perceives a reduced amount of urgency to void as compared to the urgency perceived by a subject that has not been administered or treated with an M1-selective muscarinic agonist.

As used herein, the term “decreased bladder pain” refers to a subject that perceives a reduced amount of bladder pain during, before, or after urination as compared to the bladder pain perceived by a subject during, before, or after urination that has not been administered or treated with an M1-selective muscarinic agonist.

As used herein, the term “decreased urethral pain” refers to a subject that perceives a reduced amount of urethral pain during, before, or after urination as compared to the urethral pain perceived by a subject during, before, or after urination that has not been administered or treated with an M1-selective muscarinic agonist.

As used herein, the term “decreased urinary incontinence” means a decrease in the volume of urine leakage that a subject endures between voiding as compared to the volume of urine leakage by a subject that has not been administered or treated with an M1-selective muscarinic agonist.

Throughout the present disclosure, when a particular compound is mentioned by name, for example, cevimeline, it is understood that the scope of the present disclosure encompasses pharmaceutically acceptable salts, esters, amides, or prodrugs of the named compound. In addition, if the named compound comprises a chiral center, the scope of the present disclosure also includes compositions comprising the racemic mixture of the two enantiomers, as well as compositions comprising each enantiomer individually substantially free of the other enantiomer.

### II. Treatment Methods

In one aspect, a method for treating UAB is provided. The method includes administering an effective amount of a pharmaceutical composition comprising an M1-selective muscarinic agonist to a subject in need thereof. Muscarinic receptors are acetylcholine receptors that form G protein-receptor complexes in the membranes of certain cells, such as urothelial cells. There are five subtypes of muscarinic receptors, M1 -M5, and various drugs are known to be selective for one or more of the individual receptors.

M1-selective muscarinic agonists for use in the present invention include, but are not limited to, alvameline, cevimeline, talsaclidine, xanomeline, 4-[[[(3-chlorophenyl)amino]carbonyl]oxy]-N,N,N-trimethyl-2-butyn-1-aminium chloride (McN-A 343), 1-azabicyclo[2,2,2]octane, 3-(6-chloropyrazinyl)maleate (L-689,660), 5-propargyloxycarbonyl-1,4,5,6-tetrahydropyrimidine (CDD-0097), 4-n-butyl-1-[4-(2-methylphenyl)-4-oxo-1-butyl]-piperidine (AC-42), and pharmaceutically acceptable salts and esters thereof. In some embodiments, allosteric modulators may increase the sensitivity of receptor sites. In some embodiments, suitable allosteric modulators (which may also be agonists) include, but are not limited to, benzylquinolone carboxylic acid, VU-0090157, and VU-0029767. As used herein, the definition of M1-selective muscarinic agonist excludes bethanechol. In some embodiments, the M1-selective muscarinic agonist is cevimeline or cevimeline hydrochloride.

In some embodiments, underactive bladder syndrome is associated with one or more of a neurogenic disorder, a brain disease, a surgical injury, a medication, a result of aging, an infection, a psychological condition, or a physical defect. In still other embodiments, underactive bladder is not associated with any of these conditions.

In some embodiments, the method for treating underactive bladder syndrome in a subject in need thereof includes administering an effective amount of a pharmaceutical composition comprising an M1-selective muscarinic agonist to the subject, wherein the effective amount is the amount sufficient to increase bladder emptying, decrease residual urine volume in the bladder after urination, increase urine flow rate, decrease urination frequency, decrease urination urgency, decrease bladder pain, decrease urethral pain, decrease urinary incontinence, or a combination thereof. In some embodiments, the effective amount of a pharmaceutical composition comprising an M1-selective muscarinic agonist is an amount sufficient to increase one or more of bladder emptying or urine flow rate. In some embodiments, the effective amount of a pharmaceutical composition comprising an M1-selective muscarinic agonist is an amount sufficient to decrease one or more of residual urine volume in the bladder after urination, urination frequency, urination urgency, bladder pain, urethral pain, or urinary incontinence.

In some embodiments, an effective amount of the pharmaceutical composition administered according to the inventive methods may be an amount sufficient to increase bladder emptying. In a particular embodiment, the effective amount may be an amount that is sufficient to increase bladder emptying by about 25 vol % to about 300 vol %, about 25 vol % to about 200 vol %, about 25 vol % to about 100 vol %, or about 50 vol % to about 75 vol %. The increase in bladder emptying may be quantified as the amount of volume of urine emptied from the bladder of a subject as compared to the volume of urine emptied from the bladder of the same subject when not administered an effective amount of the pharmaceutical composition.

In some embodiments, the effective amount is an amount of the pharmaceutical composition administered according to the inventive methods that is sufficient to decrease the residual urine volume in the bladder of the subject after urination or a micturition attempt by the subject. In particular embodiments, the residual urine volume remaining in the bladder of the subject after attempting urination is decreased by about 10 vol % to about 100 vol %, about 10 vol % to about 90 vol %, about 20 vol % to about 80 vol %, about 30 vol % to about 70 vol %, about 40 vol % to about 60 vol %, or about 50 vol %. The decrease in residual urine volume may be quantified as the volume amount of urine remaining in the bladder of a subject after urination or a micturition attempt as compared to the volume of residual urine remaining in the bladder of the same subject after urination or a micturition attempt when not administered an effective amount of the pharmaceutical composition. See, for example, Tables II-IV herein below.

In some embodiments, the effective amount is an amount of the pharmaceutical composition administered according to the inventive methods that is sufficient to increase the urine flow rate from the bladder of a subject during urination. In a particular embodiment, the effective amount may be an amount that is sufficient to increase the urine flow rate by about 100% to about 300%, about 100% to about 200%, about 100% to about 150%, about 150% to about 250%, about 150% to about 300%, about 200% to about 300%, or about 250% to about 300%. The increase in urine flow rate may be quantified as the rate (in mL/s) of urine emptied from the bladder of a subject as compared to the rate of urine emptied from the bladder of the same subject when not administered an effective amount of the pharmaceutical composition. See, for example, Tables II-IV herein below.

In some embodiments, the pharmaceutical composition comprising the M1-selective muscarinic agonist is administered according to a dosage regimen. In some embodiments, the dosage regimen is about 60 mg to about 90 mg of the pharmaceutical composition taken three or four time per day. In some embodiments, the pharmaceutical composition is administered on an as-needed basis.

In some embodiments, the pharmaceutical composition administered according to the present invention further comprises a rapid-release component or a sustained-release component. In some embodiments, the pharmaceutical composition comprises a rapid release component and delivers a maximal concentration of the M1-selective muscarinic agonist into the subject in about 5 minutes to about 100 minutes.

In another aspect, a method for decreasing residual urine volume in a subject after urination is provided. The method comprises administering an effective amount of a pharmaceutical composition comprising an M1-selective muscarinic agonist to the subject. In some embodiments, the M1-selective muscarinic agonist comprises cevimeline. In another aspect, the residual urine volume is decreased by about 10 vol % to about 100 vol %.

In another aspect, a method for increasing bladder emptying in a subject is provided. The method comprises administering an effective amount of a pharmaceutical composition comprising an M1-selective muscarinic agonist to the subject. In some embodiments, the M1-selective muscarinic agonist is cevimeline. In other embodiments, the bladder emptying is increased by about 25 vol % to about 300 vol %.

In yet another aspect, a method for increasing a urine flow rate in a subject is provided. The method comprises administering an effective amount of a pharmaceutical composition comprising an M1-selective muscarinic agonist to the subject. In some embodiments, the M1-selective muscarinic agonist is cevimeline. In other embodiments, the urine flow rate is increased by about 100% to about 300%.

Suitable routes of administration of the pharmaceutical composition described herein include, for example, oral, transdermal, rectal, transmucosal, or intestinal administration; parenteral delivery, including intramuscular, subcutaneous, intravenous, intramedullary injections, as well as inhalation, intrathecal, direct intraventricular, intraperitoneal, intranasal, or intraocular injections. Alternately, one may administer the pharmaceutical composition in a local rather than a systemic manner, for example, via injection of the pharmaceutical composition directly in the renal or groin area, often in a sustained, extended, or delayed release formulation. In addition, one may administer the pharmaceutical composition by a transdermal approach. In some embodiments, the step of administering is oral, transdermal, rectal, or intravenous.

In some embodiments, the invention provides a pharmaceutical composition for injection containing a compound of the present invention and a pharmaceutical excipient suitable for injection. Components and amounts of agents in the compositions are as described herein.

The forms in which the compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques that yield a powder of the active ingredient plus any additional desired ingredient from a previously sterile-filtered solution thereof.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

In some embodiments, the pharmaceutical composition is administered to a subject orally. Examples of oral administration include, but are not limited to, swallowing a pill, a tablet, caplet, a capsule, and the like. For oral administration, the M1-selective muscarinic agonist can be formulated readily by combining the active compounds with pharmaceutically acceptable carriers well known in the art. Such carriers enable the compounds to be formulated as tablets, pills, capsules, liquids, gels, syrups, slurries, suspensions, and the like, for oral ingestion by a subject. Pharmaceutical compositions for oral use can be obtained by mixing one or more solid excipients with the compound, optionally grinding the resulting mixture, and processing the mixture of granules, after adding suitable auxiliaries, if desired, to obtain tablets. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). Disintegrating agents may be added, such as the cross-linked polyvinyl pyrrolidone, agar, or alginic acid or a salt thereof such as sodium alginate.

In some embodiments, the pharmaceutical composition may be a liquid pharmaceutical composition suitable for oral consumption. Pharmaceutical compositions suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms can be prepared using anhydrous or low moisture containing ingredients and low moisture or low humidity conditions. Pharmaceutical compositions and dosage forms that contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions to provide tablets that disintegrate when exposed to an aqueous environment. Too much of a disintegrant may produce tablets that may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethylaureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the essential active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example, peanut oil, liquid paraffin, or olive oil.

Surfactants that can be used to form pharmaceutical compositions and dosage forms include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10 oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, .epsilon.-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, .epsilon.-caprolactone and isomers thereof, .delta.-valerolactone and isomers thereof, .beta.-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer may be present in an amount of about 1% to about 100%, more typically about 5% to about 25% by weight.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylaniine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, active ingredients across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

In some embodiments, the invention provides a pharmaceutical composition for transdermal delivery containing a compound of the present invention and a pharmaceutical excipient suitable for transdermal delivery. An exemplary formulation for use in the methods of the present invention employs transdermal delivery devices (“patches”). Such transdermal patches may be used to provide continuous or discontinuous infusion of a compound of the present invention in controlled amounts, either with or without another agent.

In some embodiments, the compound is administered in a single dose. A single dose of the compound may also be used for treatment of an acute condition. In another embodiment, the pharmaceutical composition is administered to the subject according to a dosage regimen, or in multiple doses. Dosing may be about once, twice, three times, four times, five times, six times, or more than six times per day. Dosing may be about once a month, once every two weeks, once a week, or once every other day. In another embodiment, two M1-selective muscarinic agonists are co-administered. In another embodiment, the administration of one or more M1-selective muscarinic agonists continues for less than about 7 days. In yet another embodiment, the administration continues for more than about 6, 10, 14, 28 days, two months, six months, or one year. In some cases, continuous dosing is achieved and maintained as long as necessary.

In some embodiments, the dosage regimen is about 30 mg, about 60 mg, or about 90 mg of the pharmaceutical composition taken one, two, three, four, or five times per day. In other embodiments, the dosing regimen is about 30 mg to about 90 mg, about 30 mg to about 60 mg, or about 60 mg to about 90 mg of the pharmaceutical composition taken 1-4, 1-3, 1-2, 2-4, 2-3, or 3-4 times per day.

An effective amount of the pharmaceutical composition may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

In other embodiments, the pharmaceutical composition is administered to the subject on an as-needed basis, e.g., to induce urination before bedtime, or before an anticipated extended time away from a bathroom.

Administration of the pharmaceutical composition may continue as long as necessary. In some embodiments, the pharmaceutical composition is administered for more than 1, 2, 3, 4, 5, 6, 7, 14, or 28 days. In some embodiments, the pharmaceutical composition is administered for less than 28, 14, 7, 6, 5, 4, 3, 2, or 1 day. In some embodiments, the pharmaceutical composition is administered chronically on an ongoing basis, e.g., for the treatment of chronic effects associated with underactive bladder syndrome.

In some embodiments, the pharmaceutical composition may further include a rapid-release component. In embodiments, the rapid-release component delivers a maximal concentration Cₘₐₓ into the subject in a time range of about 1 minute to about 100 minutes, about 5 minutes to about 100 minutes, about 5 minutes to about 60 minutes, about 10 minutes to about 60 minutes, about 10 minutes to about 30 minutes, about 10 minutes to about 20 minutes, about 20 minutes to about 30 minutes, or about 20 minutes. In some embodiments, the pharmaceutical composition that includes the rapid release component is cevimeline.

In other embodiments, the pharmaceutical composition further includes a sustained-release component. In some embodiments, the sustained-release component is configured to deliver a steady-state concentration of the pharmaceutical composition to the subject over a period of about 1 hour to about 24 hours, about 1 hour to about 18 hours, about 1 hour to about 12 hours, or about 1 hour to about 6 hours. In some embodiments, the pharmaceutical composition that includes the sustained-release component is cevimeline.

In various embodiments, the pharmaceutical composition interacts systemically with one or more urothelial cell M1 muscarinic receptors in the bladder of the subject. In certain embodiments, the pharmaceutical composition interacts locally with one or more urothelial cell M1 muscarinic receptors in the bladder of the subject. In yet other embodiments, the pharmaceutical composition may interact both locally and systemically with one or more urothelial cell M1 muscarinic receptors of the subject. In still other embodiments, the pharmaceutical composition may interact with sensory and/or motor nerve cells in or associated with the bladder or bladder function.

In particular embodiments, the M1-selective muscarinic agonist is cevimeline or a pharmaceutically acceptable salt or ester of cevimeline. As used herein, cevimeline includes (2R,5R)-2-methylspiro[1,3-oxathiolane-5,3′-1-azabicyclo[2.2.2]octane] and/or (5R)-2-methylspiro[1,3-oxathiolane-5,3′-1-azabicylo[2.2.2]octane]. In some embodiments, the M1-selective muscarinic agonist is cevimeline hydrochloride.

In other embodiments, the M1-selective muscarinic agonist may be cevimeline or a derivative of cevimeline represented by the general formula (I):

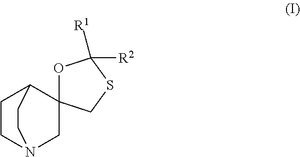

(wherein R¹ and R² may be the same or different, and independently represent a hydrogen atom, an alkyl group, a cyclopentyl group, a cyclohexyl group, a monoaryl- or diaryl-substituted methylol group, or an aryl-substituted alkyl group) or an acid salt or ester thereof. In still other embodiments, the M1-selective muscarinic agonist may be a prodrug formulation of cevimeline. Alternatively, in some embodiments cevimeline and/or derivatives of cevimeline may themselves be prodrugs for an M1-selective muscarinic agonist that is delivered locally to the bladder.

In another aspect, another method for treating UAB is provided. The method includes administering a pharmaceutical composition comprising cevimeline (or a pharmaceutically acceptable salt or ester of cevimeline) to a subject in an effective amount. Pharmaceutical compositions suitable for use in the present invention include compositions where the active ingredients are contained in an amount effective to achieve its intended purpose. More specifically, an effective amount may mean an amount of cevimeline effective to prevent, alleviate, or ameliorate one or more symptoms of UAB as described herein.

In some embodiments, an effective amount of cevimeline may be an amount sufficient to increase bladder emptying. In some embodiments, the effective amount is an amount of cevimeline that is sufficient to significantly decrease the residual urine volume in the subject after urination or a micturition attempt by the subject. In some embodiments, an effective amount of cevimeline may be an amount sufficient to increase the rate of urine flow from the bladder of a subject during urination.

In another embodiment, cevimeline is administered to the subject according to a dosage regimen. In some embodiments, the dosage regimen is about 30 mg, about 60 mg, or about 90 mg of cevimeline taken one, two, three, four, or five times per day. In some embodiments, the dosage regimen is about 30 mg to about 90 mg, about 60 mg to about 90 mg, or about 30 mg to about 60 mg taken 1-2, 1-3, 1-4, 2-3, 2-4, or 3-4 times per day. In other embodiments cevimeline is administered to the subject on an as-needed basis, e.g., to induce urination before bedtime.

In still another aspect, a method of inducing micturition is provided. The method includes administering an M1-selective muscarinic agonist in an effective amount to a subject. In certain embodiments, the M1-selective muscarinic agonist interacts with one or more urothelial cell M1 receptors in the subject. In some embodiments, the M1-selective muscarinic agonist may be cevimeline or a pharmaceutically acceptable salt or ester of cevimeline.

### III. EXAMPLES

Six different patients who each suffered from one or more medical conditions and symptoms of UAB syndrome were examined. UAB symptom-related data obtained from the patients before and after treatment with cevimeline was obtained from some of the patients and is presented below in Tables I-IV.

TABLE I
Patient Information

| Patient No. | Condition(s) | Age |
|---|---|---|
| 1 | Chrome obstructive pulmonary | 72 |
| | disease, hypertension | |
| 2 | Coronary artery disease, | 82 |
| | hypertension | |
| 3 | Breast cancer, thalassemia | 56 |
| 4 | Diabetes mellitus, hematuria, | 85 |
| | Alzheimer's Disease | |
| 5 | Renal impairment | 73 |
| 6 | Hypertension | 80 |

TABLE II
Baseline Measurements

| Patient No. | Qₘₐₓ (mL/s) | PVR (mL) | UAB Score |
|---|---|---|---|
| 1 | 6.3 | 169 | 9 |
| 2 | 4 | 500 | 13 |
| 3 | — | — | 9 |
| 4 | 3 | 200 | 9 |
| 5 | 3.4 | 99 | 7 |
| 6 | 3 | 120 | 10 |

TABLE III
Observations after one week of treatment with cevimeline (30 mg tid).

| Patient No. | Qₘₐₓ (mL/s) | PVR (mL) | UAB Score |
|---|---|---|---|
| 1 | — | — | 9 |
| 2 | — | — | — |
| 3 | — | — | — |
| 4 | 3 | 240 | 9 |
| 5 | — | — | — |
| 6 | 2.9 | 28 | 12 |

TABLE IV
Observations after two weeks of treatment with cevimeline (60 mg tid).

| Patient No. | Qₘₐₓ (mL/s) | PVR (mL) | UAB Score |
|---|---|---|---|
| 1 | — | — | — |
| 2 | — | — | — |
| 3 | — | — | — |
| 4 | 9.5 | 92 | 7 |
| 5 | — | — | — |
| 6 | 9.9 | 69 | 7 |

Table I illustrates the physical condition of each patient that was subject to treatment with cevimeline. Patients each had one or more medical conditions that were believed to have caused UAB syndrome or that may have resulted in symptoms of UAB syndrome. The patients ranged from 56 years of age to 85 years of age.

Table II shows the baseline measurements for the patients prior to receiving cevimeline treatment. Quantitative measurements were made to determine maximum urine flow rate Qₘₐₓ (expressed in mL/s) and residual urine volume in the bladder PVR (expressed in mL). Qualitative measurements were obtained based on the patient's perceived UAB symptoms. A lower UAB score indicates an improvement in UAB symptoms.

Table III shows the data obtained from patient nos. 4 and 6 after one week of treatment with cevimeline. Cevimeline was administered to each patient in 30 mg doses, three times per day. Patient 4 did not demonstrate any quantitative or qualitative improvement in UAB symptoms. Patient 6, however, did show quantitative improvement after one week of treatment. Specifically, the volume of residual urine in the bladder of patient 6 decreased from a baseline volume of 120 mL to a treated volume of 28 mL.

To assess for qualitative improvement of UAB symptoms upon administration of cevimeline, patients were surveyed using a five-question UAB symptom questionnaire. The UAB score was then tallied from the patient responses.

Table IV shows the data obtained from patient nos. 4 and 6 after a two-week treatment with cevimeline. Cevimeline was administered to each patient in 60 mg doses, three times per day. Patient 4 demonstrated both quantitative and qualitative improvements of UAB syndrome-related symptoms. For instance, patient 4 had a baseline maximum urine flow rate of 3 mL/s that was improved to 9.5 mL/s after cevimeline treatment. Patient 4 had a baseline residual urine volume of 200 mL that decreased to a residual urine volume of 92 mL after cevimeline treatment. Additionally, patient 4 had qualitative improvements in UAB symptoms, suggesting an improved quality of life after treatment with cevimeline.

Similarly, patient 6 demonstrated both quantitative and qualitative improvements of UAB syndrome-related symptoms after treatment with cevimeline. Patient 6 had a baseline maximum urine flow rate of 3 mL/s that improved to 9.9 mL/s after treatment with cevimeline. Patient 6 had a baseline residual urine volume of 120 mL that improved to a residual urine volume of 69 mL after cevimeline treatment. Furthermore, patient 6 also showed a qualitative improvement in UAB symptoms.

This initial data obtained from preliminary studies indicated that cevimeline was effective at treating the symptoms related to UAB syndrome and was associated with an improvement of the quality of life for patients suffering from symptoms related to UAB syndrome.

While some embodiments have been particularly shown and described with reference to the foregoing alternative embodiments, it should be understood by those skilled in the art that various alternatives to the embodiments described herein may be employed in practicing the invention without departing from the spirit and the scope of the invention as defined by the following claims. It is intended that the following claims define the scope of the invention and that methods and systems within the scope of these claims and their equivalents be covered thereby. This description of some embodiments should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element or the equivalent thereof, such claims should be understood to include incorporation of one or more such elements, neither requiring nor excluding two or more such elements.

We claim:

1. A method for treating an underactive bladder syndrome in a subject in need thereof, comprising administering an effective amount of a pharmaceutical composition comprising an M1-selective muscarinic agonist to the subject.

2. The method of claim 1, wherein the underactive bladder is associated with one or more of:
a neurogenic disorder;
a brain disease;
a surgical injury;
a medication;
a result of aging;
an infection;
a psychological condition; or
a physical defect.

3. The method of claim 1, wherein the effective amount is an amount sufficient to increase bladder emptying, decrease residual urine volume in the bladder after emptying, increase urine flow rate, decrease urination frequency, decrease urination urgency, decrease bladder pain, decrease urethral pain, decrease urinary incontinence, or a combination thereof.

4. The method of claim 1, wherein the M1-selective muscarinic agonist is selected from the group consisting of alvameline, cevimeline, talsaclidine, xanomeline, McN-A 343, L-689,660, CDD-0097, ACT-42, and combinations thereof.

5. The method of claim 4, wherein the M1-selective muscarinic agonist comprises cevimeline.

6. The method of claim 3, wherein the administering is according to a dosage regimen of about 60 mg to about 90 mg of the pharmaceutical composition taken three or four times per day.

7. The method of claim 3, wherein the effective amount is the amount sufficient to decrease the residual urine volume in the bladder after urination and the residual urine volume is decreased by about 10 vol % to about 100 vol %.

8. The method of claim 3, wherein the effective amount is the amount sufficient to increase bladder emptying and the bladder emptying is increased by about 25 vol % to about 300 vol %.

9. The method of claim 3, wherein the effective amount is the amount sufficient to increase urine flow rate and the urine flow rate is increased by about 100% to about 300%.

10. The method of claim 1, wherein the pharmaceutical composition further comprises a rapid-release component or a sustained release component.

11. The method of claim 10, wherein the pharmaceutical composition comprises a rapid release component and the rapid release component delivers a maximal concentration Cₘₐₓ into the subject in about 5 minutes to about 100 minutes.

12. The method of claim 1, wherein the pharmaceutical composition is administered on an as-needed basis.

13. A method for decreasing a residual urine volume in a subject after urination comprising administering an effective amount of a pharmaceutical composition comprising an M1-selective muscarinic agonist to the subject.

14. The method of claim 13, wherein the M1-selective muscarinic agonist comprises cevimeline.

15. The method of claim 13, wherein the residual urine volume is decreased by about 10 vol % to about 100 vol %.

16. A method for increasing bladder emptying in a subject comprising administering an effective amount of a pharmaceutical composition comprising an M1-selective muscarinic agonist to the subject.

17. The method of claim 16, wherein the M1-selective muscarinic agonist comprises cevimeline.

18. The method of claim 16, wherein the bladder emptying is increased by about 25 vol % to about 300 vol %.

19. A method for increasing a urine flow rate in a subject comprising administering an effective amount of a pharmaceutical composition comprising an M1-selective muscarinic agonist to the subject.

20. The method of claim 19, wherein the M1-selective muscarinic agonist comprises cevimeline.

21. The method of claim 19, wherein the urine flow rate is increased by about 100% to about 300%.
